(19) United States
(12) Patent Application Publication
(10) Pub. No.: US 2007/0082854 A1
(43) Pub. Date: Apr. 12, 2007

(54) Novel polymorphs of erythromycin compound

(75) Inventors: Pandurang Balwant Deshpande, Vadodara (IN); Parven Kumar Luthra, Vadodara (IN); Manish Kanchanbhai Patel, Vadodara (IN); Mahesh Pravinchandra Davadra, Vadodara (IN)

Correspondence Address: NIXON & VANDERHYE, PC, 901 NORTH GLEBE ROAD, 11TH FLOOR, ARLINGTON, VA, 22203, US

(73) Assignee: ALEMBIC LIMITED, Vadodara (IN)

(21) Appl. No.: 11/528,396

(22) Filed: Sep. 28, 2006

(30) Foreign Application Priority Data
Oct. 6, 2005 (IN) 1260/MUM/2005

(51) Int. Cl. A61K 31/7052; C07H 17/08

(52) U.S. Cl. 514/29; 536/7.4

(57) ABSTRACT

The present invention relates to novel polymorphs of Telithromycin designated as Form I, II and III and its process for preparation. Also it provides Telithromycin with at least 99% purity.

### FIELD OF THE INVENTION

The present invention relates to novel polymorphs of Telithromycin of formula (I) and process for its preparation. These novel polymorphs are designated as Form I, Form II and Form III. The present invention also provides Telithromycin having Organic Volatile Impurities (referred as OVI hereinafter) less than 1% w/w. Present invention also provides Telithromycin having purity at least 99%.

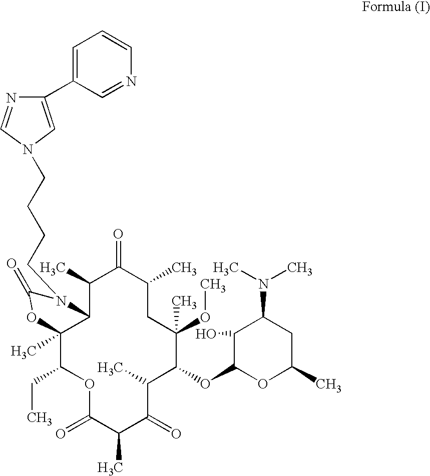

### BACKGROUND OF THE INVENTION

Telithromycin is chemically known as 11,12-dideoxy-3-de[(2,6-dideoxy-3-C-methyl -3-O-methyl-α-L-ribohexopyranosyl)oxy]-6-O-methyl-3-oxo-12,11 - (oxycarbonyl[4-[4-(3-pyridinyl)-1 H-imidazol-1-yl]butyl]imino)-erythromycin. It is marketed under brand name “Ketek” and is prescribed for the treatment of bacterial infections. Telithromycin of formula (I) is a ketolide which differs chemically from the macrolide group of antibacterials by the lack of α-L-cladinose at position 3 of the erythronolide A ring, resulting in a 3-keto function. It is further characterized by a C₁₁-C₁₂ carbamate substituted by an imidazolyl and pyridyl ring through a butyl chain. Telithromycin exhibits antibacterial activity and is used for treatment of community acquired pneumonia, acute exacerbation of chronic bronchitis, acute sinusitis, tonsillitis/pharyngitis.

Polymorphism is the occurrence of different crystalline forms of a single compound and it is a property of some compounds and complexes in solid state. The polymorphic and pseudopolymorphic solids display different physical properties, including those due to packing, and various thermodynamic, spectroscopic, interfacial and mechanical properties (See H. Brittain, Polymorphism in Pharmaceutical Solids, Marcel Dekker, New York, N.Y., 1999, pp. 1-2). Polymorphic and pseudopolymorphic forms of the drug substance (also known as the “active pharmaceutical ingredient” (API)), as administered by itself or formulated as a drug product (also known as the final or finished dosage form, or as the pharmaceutical composition) are well known and may affect, for example, the solubility, stability, flowability, tractability, and compressibility of drug substances and the safety and efficacy of drug products, (see, e.g., Knapman, K Modem Drug Discoveries, March 2000: 53). Polymorphs of a compound can be characterized by x-ray diffraction pattern, infrared spectrum, DSC etc.

One important physical property that can vary between two polymorphic forms is solubility, which can affect the bioavailability of the drug. Therefore there is a need to develop new polymorphic forms of a drug since it provides new opportunity to improve the performance characteristics of a pharmaceutical product.

Telithromycin was first reported in U.S. Pat. No. 5,635,485, which disclose its process for preparation. The inventors of present invention have unexpectedly found that Telithromycin exhibits different polymorphic forms. These novel forms are characterized as Form I, Form II and Form III.

The solvents traditionally used for the preparation of a drug substance are sometimes difficult to remove completely by practical manufacturing techniques, which are in actuality employed during the production. Therefore, in the preparation of drug substance wherein plural steps are serially carried out till the final step, each solvent used in each step may possibly residue in drug substance. Further the residual solvents in drug substances may alter it biological activity.

Since a solvent may play an important role in increasing the yield rate or in determination of physical properties of drug substance such as crystal form, purity, solubility, etc., even if such a solvent is known to be toxic, there may be many cases that the use thereof in the preparation of drug substance cannot be avoided in terms of risk-benefits. In such cases, this guideline (ICH guidelines Q3C(R3)) decrees that a concentration of a residual solvent in drug substance should be not more than a specified value, which is toxicologically acceptable.

The ICH Q3A(R1) guidance for API manufacturers requires that process impurities should be maintained below set limits by controlling process parameters, such as temperature, pressure, time, and stoichiometric ratios, and including purification steps, such as crystallization, distillation, and liquid-liquid extraction, in the manufacturing process.

It was observed by the present inventor after the analysis of the drug product of the innovators sample that the content of the epimeric impurity is above 1% w/w which is contrary to ICH guidelines for the content of impurity. Therefore it is also an object of the present invention to provide Telithromycin having content of epimeric impurity below 1% w/w.

Further it was observed by the inventors that the process of the present invention is unexpectedly advantageous for the commercial scale production of Telithromycin of formula (I) with high yield, high purity, and low value residual solvent. The process is more economic in addition to being eco-friendly.

### OBJECT OF THE INVENTION

The primary object of the present invention is to provide novel polymorphs of Telithromycin of formula (I).

Another object of the invention is to provide novel polymorphs of Telithromycin of formula (I) designated as Form I, Form II and Form III.

Yet another object of the invention is to provide process for preparation of novel polymorphs of Telithromycin of formula (I) designated as Form I, Form II and Form III.

Another object of the invention is to provide Telithromycin having purity at least 99%.

A further object of present invention is to provide Telithromycin having OVI less than 1% w/w.

Yet another object of the invention is to provide Telithromycin having particle size wherein d(0.5) is less than or equal to about 5 μm and d(0.9) is less than or equal to about 10 μm.

### BRIEF DESCRIPTION OF THE DRAWINGS

FIG. 1 represents PXRD of Telithromycin Form I.

FIG. 2 represents PXRD of Telithromycin Form II.

FIG. 3 represents PXRD of Telithromycin Form III.

### SUMMARY OF THE INVENTION

In accordance with the object of the present invention, one aspect provides novel polymorphs of Telithromycin designated as Form I, Form II and Form III.

Another aspect of the present invention provides Telithromycin Form I, characterized by powder x-ray diffraction peaks at 6.0, 9.6, 11.1, 11.4, 13.3, 19.6 ±0.2° 2−θ values.

Yet another aspect of the present invention provides Telithromycin Form II, characterized by powder x-ray diffraction peaks at 7.7, 10.0, 12.0, 12.9, 15.8, 18.8±0.2° 2−θ values.

The further aspect of the present invention provides Telithromycin Form III, characterized by powder x-ray diffraction peaks at 8.2, 10.4, 12.6, 15.7, 16.9±0.2° 2−θ values.

Yet another aspect of the present invention provides process of the preparation of novel polymorphs of Telithromycin designated as Form I, Form II and Form III.

The further aspect of the present invention provides Telithromycin having OVI less than 1% w/w.

Accordingly yet another aspect of the present invention provides Telithromycin having purity at least 99%.

Another aspect of the present invention provides Telithromycin having epimeric impurity less than 1% w/w.

Yet another aspect of the present invention is to provide Telithromycin having particle size wherein d(0.5) is less than or equal to about 5 μm and d(0.9) is less than or equal to about 10 μm.

### DETAILED DESCRIPTION OF THE INVENTION

The term “treating” as used herein above refers to simple dictionary meaning: “To subject to a process, action, or change, especially to a chemical or physical process or application”. It is also indented to include chemical processes such as leaching, slurring, contacting and the like.

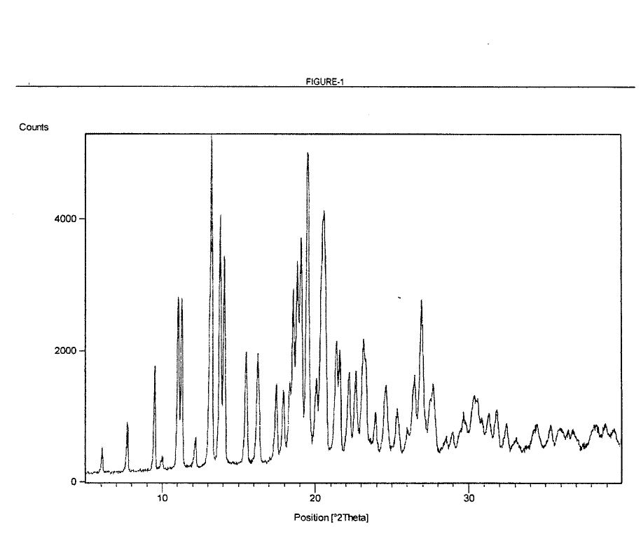

A preferred embodiment of the present invention provides Telithromycin Form I which is characterized by powder x-ray diffraction spectrum which is substantially the same as shown in FIG. 1.

Telithromycin Form I is characterized by powder x-ray diffraction peaks at 6.0, 9.6, 11.1, 11.4, 13.3, 19.6±0.2° 2−θ values. It is further characterized by powder x-ray diffraction peaks at 7.7, 10.0, 13.9, 14.1, 15.5, 16.3, 17.5, 18.0, 18.6, 18.9, 19.2, 20.6, 27.0±0.20° 2−θ values.

The process for the preparation of Telithromycin Form I comprises steps of,

(a) treating Telithromycin with halogenated solvent

(b) treating the solution obtained in step (a) with an anti-solvent

Telithromycin is treated with halogenated solvent at temperature of about 20° C. to about boiling temperature of the solvent, preferably at about room temperature, to obtain a solution. Further this solution is treated with an anti-solvent and stirred for about 1 hour to about 5 hours at temperature of about 20° C. to about 35° C., preferably at about room temperature to obtain Telithromycin Form I. Telithromycin Form I can be further isolated by conventional methods such as filtration or centrifugation and dried.

The halogenated solvent as mentioned hereinabove is selected from group comprising of methylenedichloride, ethylene dichloride, chloroform, carbon tetrachloride and the like or mixtures thereof. The preferred solvent is methylenedichloride. The examples of anti-solvent as mention hereinabove includes, but is not limited to methyltertbutyl ether, diethyl ether, diisopropyl ether, cyclohexane, n-heptane, n-hexane and the like or mixtures thereof. The preferred one is methyltertbutyl ether.

Telithromycin Form I can also be prepared from Telithromycin Form II, Form III or mixtures thereof by the general process described above for the preparation of Form I.

It was observed by the inventors of present invention that Form I of Telithromycin is solvated form which contains mixture of solvents entrapped in its crystal lattice.

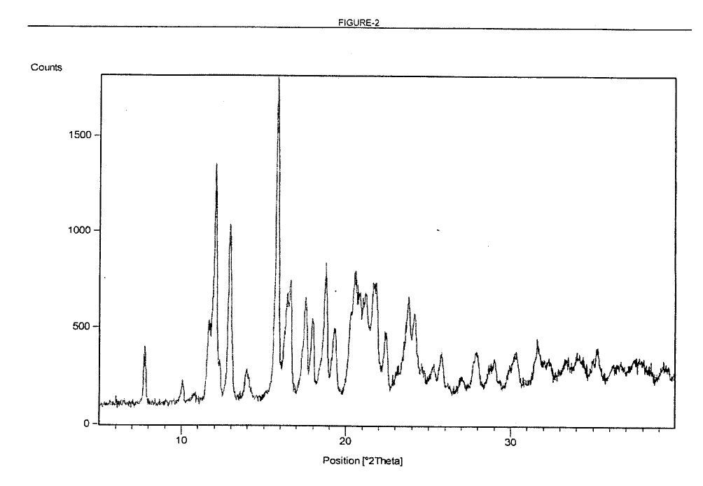

Another embodiment of the present invention provides Telithromycin Form II which is characterized by powder x-ray diffraction spectrum which is substantially the same as shown in FIG. 2.

Telithromycin Form II is characterized by powder x-ray diffraction peaks at 7.7, 10.0, 12.0, 12.9, 15.8, 18.8±0.2° 2−θ values. It is further characterized by powder x-ray diffraction peaks at 11.6, 16.6, 17.5, 17.9, 18.8, 19.3, 20.5, 21.2, 21.8±0.2° 2−θ values.

Telithromycin Form II is prepared by process comprising treating Telithromycin with solvent selected from the group comprising of C₁₋₈ ester, C₄₋₈ cycloalkane, C₂₋₁₂ ether, C₅₋₁₂ saturated hydrocarbon, C₁₋₆ ketone and the like or mixtures thereof.

Telithromycin is treated with solvent selected from the group comprising of C₁₋₈ ester, C₄₋₈ cycloalkane or C₂₋₁₂ ether, C₅₋₁₂ saturated hydrocarbon and C₁₋₆ ketone or mixtures thereof at temperature of about 20° C. to about boiling temperature of the solvent, preferably at about room temperature, for about 1 hour to about 10 hours, preferably for about 6 hours to about 8 hours to obtain Telithromycin Form II. If desired the reaction mass may be cooled to about 15° C. to 25° C. Telithromycin Form II can be isolated by conventional methods such as filtration or centrifugation and dried.

The C₁₋₈ ester as mentioned hereinabove is selected from group comprising of ethyl acetate, butyl acetate, methyl acetate and the like or mixtures thereof. The preferred solvent is ethyl acetate. The C₄₋₈ cycloalkane as mentioned hereinabove is selected from group comprising of cyclohexane, cycloheptane, cyclopentane and the like or mixtures thereof. The preferred solvent is cyclohexane. The C₂₋₁₂ ether as mentioned hereinabove is selected from group comprising of diethyl ether, diisopropyl ether, tetrahydrofuran and the like or mixtures thereof. C₅₋₁₂ saturated hydrocarbon as mentioned hereinabove is selected from group comprising of n-heptane, n-hexane, n-pentane and the like, or mixtures thereof. The preferred one is n-heptane. The C₁₋₆ ketone as mentioned hereinabove is selected from group comprising of acetone, methyl ethyl ketone, methyl isobutyl ketone and the like or mixtures thereof. The preferred one is acetone.

Telithromycin Form II can also be prepared from Telithromycin Form I, Form III or mixtures thereof by the general process described above for the preparation of Form II.

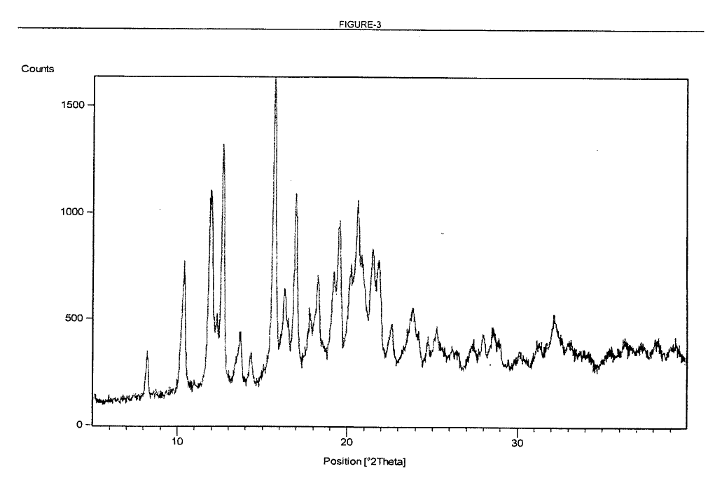

Further embodiment of the present invention provides Telithromycin Form III which is characterized by powder x-ray diffraction spectrum which is substantially the same as shown in FIG. 3.

Telithromycin Form III is characterized by powder x-ray diffraction peaks at 8.2, 10.4, 12.6, 15.7, 16.9±0.20° 2−θ values. It is further characterized by powder x-ray diffraction peaks at 12.0, 13.7, 16.3, 18.3, 20.6, 21.5, 21.9±0.2° 2−θ values.

Telithromycin Form III is prepared by process comprising treating Telithromycin with solvent selected from the group comprising of C₄₋₈ cycloalkane in the presence of aromatic hydrocarbon to obtain Telithromycin Form III

Telithromycin is treated with solvent selected from the group comprising of C₄₋₈ cycloalkane at temperature of about 20° C. to about boiling temperature of the solvent, preferably at about room temperature, in the presence of aromatic hydrocarbon, for about 1 hour to about 10 hours, preferably for about 6 hours to about 8 hours to obtain Telithromycin Form III. If desired the reaction mass can be cooled to about 15° C. to 25° C. Telithromycin Form III can be isolated by conventional methods such as filtration or centrifugation and dried.

The C₄₋₈ cycloalkane as mentioned hereinabove is selected from group comprising of cyclohexane, cycloheptane, cyclopentane and the like or mixtures thereof. The preferred solvent is cyclohexane. The examples of aromatic hydrocarbon as mentioned hereinabove includes but is not limited to toluene, benzene and the like or mixtures thereof. The preferred one is toluene.

Telithromycin Form III can also be prepared from Telithromycin Form I, Form II or mixtures thereof by the general process described above for the preparation of Form III.

Yet another embodiment of the present invention provides Telithromycin having purity at least 99%.

Further embodiment of the present invention provides Telithromycin having epimeric impurity less than 1% w/w, preferably less than 0.5%, more preferably less than 0.2%.

Still another embodiment of the present invention provides Telithromycin having OVI less than 1% w/w, preferably less than 0.5% w/w and more preferably less than 0.1% w/w.

Yet another embodiment of the present invention provides Telithromycin having particle size wherein d(0.5) is less than or equal to about 5 μm d(0.9) is less than or equal to about 10 μm.

The process of the present invention is illustrated by the following examples, which are illustrative only and should not be construed so as to limit the scope of the invention in any manner.

### EXAMPLES

Telithromycin used in the process of preparations given below in examples, can be prepared by methods known perse or by any methods known to person skilled in art, particularly by process disclosed in the co-pending PCT application published as WO2005105821.

### Example 1

### Process for Preparing Telithromycin Form I

5 ml dichloromethane was added to 10 g of Telithromycin to obtain clear solution. 100 ml of methyltertbutyl ether is added and reaction mass was stirred for about 3 hours at 25° C. to 30° C. The solid was filtered and washed with methyltertbutyl ether and dried at 25° C. to 30° C. under vacuum to obtain Form-I.

### Example 2

### Process for Preparing Telithromycin Form II

10 g Telithromycin Form I prepared in Example 1 is taken in 80 ml mixture of ethyl acetate and n-heptane. The reaction mixture is refluxed at 80° C. for about 6 to 8 hours and then cooled to about 15° C. The product is filtered, washed and dried in vacuum at 40° C. to obtain Telithromycin Form II (purity: 99.3%, epimeric impurity: 0.26%, OVI: 0.2%)

### Example 3

### Process for Preparing Telithromycin Form III

10 g Telithromycin Form I prepared in Example 1 is taken in 80 ml mixture of cyclohexane and toluene. The reaction mixture is stirred at about 25° C. to about 30° C. for about 6 to 8 hours. The product is filtered, washed and dried in vacuum at 40° C. to obtain Telithromycin Form III (purity: 99.05%, epimeric impurity: 0.40%, OVI: 0.12%)

While the present invention has been described in terms of its specific embodiments, certain modifications and equivalents will be apparent to those skilled in the art and are intended to be included within the scope of the present invention.

1. Telithromycin Form I.

2. Telithromycin Form I characterized by powder x-ray diffraction peaks at 6.0, 9.6, 11.1, 11.4, 13.3, 19.6±0.2° 2−θ values.

3. Telithromycin Form I characterized by powder x-ray diffraction spectrum which is substantially the same as shown in FIG. 1.

4. A process for preparation of Telithromycin Form I comprises steps of,
(a) treating Telithromycin with halogenated solvent
(b) treating the solution obtained in step (a) with an anti-solvent

5. A process as claimed in claim 4, wherein said halogenated solvent is selected from group comprising of methylenedichloride, ethylene dichloride, chloroform and carbon tetrachloride.

6. A process as claimed in claim 4,. wherein said anti-solvent is selected from group comprising of methyltertbutyl ether, diethyl ether, diisopropyl ether, cyclohexane, n-heptane and n-hexane.

7. Telithromycin Form II.

8. Telithromycin Form II characterized by powder x-ray diffraction peaks at 7.7, 10.0, 12.0, 12.9, 15.8, 18.8±0.2° 2−θ values.

9. Telithromycin Form II characterized by x-ray diffraction spectrum which is substantially the same as shown in FIG. 2.

10. A process for preparation of Telithromycin Form II comprising, treating Telithromycin with solvent selected from the group comprising of C₁₋₈ ester, C₄₋₈ cycloalkane or C₂₋₁₂ ether, C₅₋₁₂ saturated hydrocarbon and C₁₋₆ ketone or mixtures thereof to obtain Telithromycin Form II.

11. A process as claimed in claim 10, wherein said C₁₋₈ ester is selected from group comprising of ethyl acetate, butyl acetate and methyl acetate.

12. A process as claimed in claim 10, wherein said C₁₋₄₋₈ cycloalkane is selected from group comprising of cyclohexane, cycloheptane and cyclopentane.

13. A process as claimed in claim 10, wherein said C₂₋₁₂ ether is selected from group comprising of diethyl ether, diisopropyl ether and tetrahydrofuran.

14. A process as claimed in claim 10, wherein said C₅₋₁₂ saturated hydrocarbon is selected from group comprising of n-heptane, n-hexane and n-pentane.

15. A process as claimed in claim 10, wherein said C₁₋₆ ketone is selected from group comprising of acetone, methyl ethyl ketone and methyl isobutyl ketone.

16. Telithromycin Form III.

17. Telithromycin Form III characterized by powder x-ray diffraction peaks at 8.2, 10.4, 12.6, 15.7, 16.9±0.2° 2−θ values.

18. Telithromycin Form III characterized by powder x-ray diffraction spectrum which is substantially the same as shown in FIG. 3.

19. A process for preparation of Telithromycin Form IlIl comprising, treating Telithromycin with solvent selected from the group comprising of C₄₋₈ cycloalkane in presence of aromatic hydrocarbon to obtain Telithromycin Form III

20. A process as claimed in claim 19, wherein said C₄₋₈ cyloalkane is selected from group comprising of cyclohexane, cycloheptane and cyclopentane.

21. A process as claimed in claim 19, wherein said aromatic hydrocarbon is selected from group comprising of toluene and benzene.

22. Telithromycin having purity at least 99%.

23. Telithromycin having purity at least 99.5%.

24. Telithromycin having epimeric impurity less than 1% w/w.

25. Telithromycin of having OVI less than 1% w/w.

26. Telithromycin having particle size wherein d(0.5) is less than or equal to about 5 μm and d(0.9) is less than or equal to about 10 μm.
